(19) United States
(12) Patent Application Publication
(10) Pub. No.: US 2006/0142218 A1
(43) Pub. Date: Jun. 29, 2006

(54) Method of controlling telomere length

(76) Inventors: Kunihiro Ohta, Tokyo (JP); Takehiko Shibata, Tokyo (JP)

Correspondence Address: Chris T Mizumoto;Fish & Richardson, Suite 2800, 45 Rockefeller Plaza, New York, NY, 10111, US

(21) Appl. No.: 10/204,067

(22) PCT Filed: Feb. 14, 2001
(86) PCT No.: PCT/JP01/01024

§ 371 Date: Aug. 16, 2002

(30) Foreign Application Priority Data
Feb. 18, 2000 (JP) 2000-41929

(51) Int. Cl. A61K 48/00; C07H 21/04; C12N 9/22

(52) U.S. Cl. 514/44; 435/199; 536/23.2

(57) ABSTRACT

A method of controlling telomere length wherein the method comprises introducing into a cell a DNA encoding Mre11 protein or a DNA encoding a protein comprising Mre11 protein wherein a part of the nuclease domain, the C-terminal domain or the whole thereof is modified or deleted.

### TECHNICAL FIELD

This invention relates to a method of controlling telomere length wherein physiological activity of endogenous Mre11 protein in a eukaryotic cell is modified, a telomere length controlling agent comprising as an active component a substance which modifies physiological activity of endogenous Mre11 protein in a eukaryotic cell, and a gene therapeutic agent for telomere length-associated diseases comprising as an active component a substance which modifies physiological activity of endogenous Mre11 protein in a eukaryotic cell.

### BACKGROUND ART

Telomeres are functional constructs existing at the ends of double-stranded DNA constituting filamentous chromosomes in a eukaryotic cell. It is known that this construct is generally composed of simple repeats, and plays an important role in preventing chromosomal aberration due to chromosome fusion, pairing of homologous chromosomes during myosis, control of gene expression, and determination of forms of chromosomes in a nucleus.

In general, replication of a double-stranded DNA is carried out by synthesizing a RNA primer by primase and at an end thereof by elongation-synthesis of a DNA chain by DNA polymerase. The RNA primer is remeved by 5′-3′ exonuclease activity of the DNA polymerase, and at the same time the removed part is replaced with the DNA chain by the DNA polymerase. However, in the case of a filamentous chromosome, there is no replacement of the RNA primer at the 5′-most end of a nascent chain with a DNA chain, and thus when replication is completed, the chromosome is shortened by the size of the RNA primer. Therefore, every repeat of cell division progressively shortens the chromosomes from its end in the daughter cells, and finally the chromosomes become unstable, resulting in death of the cell. On the other hand, it has been shown that in an immortalized cell, telomerase which can elongate telomeres is highly expressed, thus telomere lengths can not be reduced. Consequently, intensive studies are made on regulators for cell life span or anticancer agents which involve the telomerase activity in their mechanisms.

However, recently it has been shown that in yeasts and mice etc. even when their telomerase genes are disrupted, it takes time for shortening their teromeres [Blasco, M. A., et al.: Cell, 91:25-34 (1997); Rudolph, K. L., et al.: Cell, 96:701-12 (1999); Herrera, E., et al.: Embo J, 18:2950-60(1999)], and that even a variant of a gene which is not directly related to telomerase activity, results in shortening or elongation of telomeres [Boulton, S. J., et al.: Embo J, 17:1819-28 (1998); Furuse, M., et al.: Embo J, 17:6412-25(1998); Wilson, S. et al.: Nucleic Acids Res, 27:2655-61 (1999)]. Thus, it is pointed out that there are potentially mechanisms other than telomerase for maintaining telomeres [Nakamura, T. M., et al.: Science, 282:493-6 (1998); Reddel, R. R., et al. A review. Biochemistry (Mosc), 62:1254-62 (1997)]. In particular, it has been reported that a Mre11-Rad50-Xrs2 complex which is known to be engaged in repair of double-stranded DNA cleavage etc. is also associated with controlling telomere length [Boulton, S. J., et al.: Embo J, 17: 1819-28 (1998); Furuse, M., et al.: Embo J, 17:6412-25(1998); Le, S., et al.: Genetics, 152:143-52 (1999)]. A Mre11-Rad50-Xrs2 (Nbsl) complex [Ajimura, M., et al.: Genetics, 133:51-66 (1993); Johzuka, K., et al.: Genetics, 139:1521-32 (1995)] is a protein complex formed with Mre11 as a core which has a strong DNA binding activity and functions as a double-stranded DNA exonuclease and a single-stranded endonuclease [Furuse, M., et al.: Embo J, 17:6412-25 (1998); Paull, T. T., et al.: Mol Cell, 1:969-79 (1998); Usui, T., et al.: Cell, 95:705-16 (1998); Trujillo, K. M., et al.: J Biol Chem, 273:21447-50 (1998)]. The complex functions as an essential enzyme to initiation reaction for repair of DNA double-stranded cleavage or chromosomeal recombination. In budding yeast, Saccharomyces cerevisiae, or fission yeast, Schizosaccharomyces pombe, when even one of the proteins constituting this enzyme-complex is absent, notable shortening of telomeres is observed [Boulton, S. J., et al.: Embo J, 17:1819-28(1998)]. Homologs of all the constituent proteins of the Mre11-Rad50-Xrs2(Nbs1) complex exist in humans [Dolganov, G. M., et al.: Mol Cell Biol, 16:4832-41 (1996); Petrini, J. H., et al.: Genomics, 29:80-6 (1995)], and it is thought that they have a common role in all eukaryotes from yeast to human.

### DISCLOSURE OF THE INVENTION

It is an object of the present invention to provide a method of controlling telomere length wherein the method comprises modifying physiological activity of endogenous Mre11 protein in a eukaryotic cell.

As a result of intensive studies based on the above subject, the inventors of the present invention have found that modifying physiological activity of endogenous Mre11 protein in a eukaryotic cell enables control of telomere length of the cell, thus accomplishing the present invention.

Namely, the present invention is a method of controlling telomere length wherein the method comprises modifying physiological activity of endogenous Mre11 protein in a eukaryotic cell. Herein, the modification of the physiological activities of the endogenous Mre11 protein is carried out by introducing into a cell a DNA encoding foreign Mre11 protein, or a DNA encoding a protein wherein the nuclease domain or the C-terminal domain of the foreign Mre11 protein is modified, in a state such that the DNA can be expressed.

Further, the present invention is a telomere length controlling agent or a gene therapeutic agent for telomere-associated diseases (e.g. melanoma, hepatoma, breast cancer, gastric cancer, brain tumor, and cell senescent diseases) comprising as an active agent a substance which modifies physiological activity of endogenous Mre11 protein in a eukaryotic cell. Herein, the substance which modifies physiological activity of endogenous Mre11 protein in the eukaryotic cell may include a DNA construct which comprises a DNA encoding foreign Mre11 protein, or a DNA encoding a protein wherein a nuclease domain or a C-terminal domain of foreign Mre11 protein is modified, in a state such that the DNA can be expressed.

In the present invention, the following protein (a) or (b) is provided as foreign Mre11 protein:
(a) a protein comprising an amino acid sequence represented by SEQ ID NO: 2 or 4; or
(b) a protein comprising an amino acid sequence which is the amino acid sequence represented by SEQ ID NO: 2 or 4 from, in, or to which one or more amino acids are deleted, substituted or added, and having physiological activity of Mre11 protein.

Further, in the present invention, the following DNA (c) or (d) is provided as the DNA encoding foreign Mre11 protein:
(c) a DNA comprising a nucleotide sequence represented by SEQ ID NO: 1 or 3; or
(d) a DNA which can hybridize with the DNA of (c) under a stringent condition and encodes a protein having physiological activity of Mre11 protein.

Furthermore, in the present invention, the following protein (e) or (f) is provided as a protein wherein a nuclease domain of the foreign Mre11 protein is modified:
(e) a protein comprising an amino acid sequence represented by SEQ ID NO: 6; or
(f) a protein comprising an amino acid sequence which is the amino acid sequence represented by SEQ ID NO: 6 from, in, or to which one or more amino acids are deleted, substituted or added, and having physiological activity (except nuclease activity) of Mre11 protein.

Moreover, in the present invention, the following DNA (g) or (h) is provided as DNA encoding a protein wherein the nuclease domain of the foreign Mre11 protein is modified:
(g) a DNA comprising a nucleotide sequence represented by SEQ ID No: 5; or
(h) a DNA which can hybridize with the DNA of (g) under a stringent condition and encodes a protein having physiological activity (except nuclease activity) of Mre11 protein.

Further, in the present invention, the following protein (i) or (j) is provided as a protein wherein a C-terminal domain of the foreign Mre11 protein is modified:
(i) a protein comprising an amino acid sequence represented by SEQ ID NO: 8; or
(j) a protein comprising an amino acid sequence which is the amino acid sequence represented by SEQ ID NO: 8 from, in or to which one or more amino acids are deleted, substituted or added, and having physiological activity (except double-stranded DNA binding activity) of Mre11 protein.

Still further, in the present invention, the following DNA (k) or (l) is provided as DNA encoding a protein wherein the C-terminal domain of the foreign Mre11 protein is modified:
(k) a DNA comprising a nucleotide sequence represented by SEQ ID NO: 7; or
(l) a DNA which can hybridize with the DNA of (k) and encodes a protein having physiological activity (except double-stranded DNA binding activity) of Mre11 protein.

The present invention will hereinafter be described in detail.

A method of controlling telomere length according to the present invention is based on modification of physiological activity of endogenous Mre11 protein in a eukaryotic cell. Herein, the term “controlling telomere length” means shortening, retaining or elongating telomere length in cells. The term “endogenous Mre11 protein” means a protein which natively exists in a cell and has physiological activity of Mre11 protein. The term “physiological activity of Mre11 protein” means at least one of the following activities: the ability of forming a complex with Rad50 protein and Xrs2 (Nbs1) protein, double-stranded DNA binding activity, double-stranded DNA exonuclease activity and single-stranded DNA endonuclease activity. The double-stranded DNA exonuclease activity and the single-stranded DNA endonuclease activity both as a whole are referred to as the nuclease activity. Further, “modification” means to improve, reduce or delete, partially or wholly physiological activity of the endogenous Mre11 protein. For example, shortening of telomeres can be carried out by introducing into a cell a DNA encoding a full length of foreign Mre11 protein or a DNA encoding a protein wherein the nuclease domain of the foreign Mre11 protein is modified, in a state such that the DNA can be expressed. Here, “a state such that the DNA can be expressed” means a state wherein the DNA is comprised in a vector together with a DNA region concerning the expression, e.g. a promoter so that a protein encoded by the DNA can be produced in the cell into which the DNA has been introduced. In contrast, the retaining or increasing of telomere length is carried out by introducing into a cell a DNA encoding a protein wherein the C-terminal domain of the foreign Mre11 protein is modified, in a state such that the DNA can be expressed. Here the term “foreign Mre11 protein” means Mre11 protein derived from outside of a target cell, and the term “modify” means that one or more amino acids is deleted, substituted or added from, in or to a functional domain.

1. Preparation of a DNA Encoding Foreign Mre11 Protein

(1) Sources of a DNA Encoding Foreign Mre11 Protein

The source of a DNA encoding foreign Mre11 protein which can be used for controlling telomere length is any organism-derived cell as long as it has a DNA encoding Mre11 protein, without particular limitation. Examples thereof include cells derived from various eukaryotes e.g. human (Homo sapiens), a mouse (Mus musculus), a South African clawed frog (Xenopus laevis), a fly (Drosophila melanogaster), Arabidopsis thaliana, Saccharomyces cerevisiae, Coprinus cinereus, Schizosaccharomyces pombe. Preferably a DNA encoding foreign Mre11 protein to be used for controlling telomere length can be derived from the same species as that of a target cell for telomere length control. In regard to Schizosaccharomyces pombe, a protein corresponding to Mre11 protein is called Rad32. For example, for controlling telomere length of human cells, it is preferable to use a DNA encoding human Mre11 protein, and for controlling the telomere lengths of Saccharomyce cerevisiae cells, it is preferable to use a DNA encoding Mre11 protein from Saccharomyce cerevisiae.

(2) Preparation of a DNA Encoding Foreign Mre11 Protein

A DNA encoding foreign Mre11 protein can be obtained by screening a clone including a DNA encoding Mre11 protein from a genome DNA library or a cDNA library which has been prepared from a cell derived from organisms described in the section (1) above, or by direct amplification by PCR using a genome DNA or a cDNA as a template.

For example, for obtaining the DNA encoding Mre11 protein by screening a cDNA library, initially mRNAs are prepared from cells of the organisms described in section (1) above, by a method such as the guanidine thiocyanate method. Next, a synthetic DNA primer including an oligo-dT sequence is hybridized, then a single-stranded cDNA is synthesized by a reverse transcriptase. Thereafter, using E coli DNA polymerase I, E coli DNA ligase and RNaseH, double-stranded cDNAs are synthesized by a conventional method. Next, after blunting cDNA ends by T4DNA polymerase, a DNA adaptor e.g. EcoRI adaptor, is added to both ends of the cDNA chains by T4DNA ligase. Then, the cDNA chains added by the DNA adaptors are inserted into a restriction site of a commercially available λ phage vector (e.g. λZAP available from Stratagene) or a plasmid vector (e.g. pGEM2 available from PromegaBiotech) in accordance with a conventional method. Thus, a group of recombinant λ phage DNAs or recombinant plasmid DNAs can be obtained.

Subsequently, in regard to the group of therecombinant λ phage DNAs, in vitro packaging is performed using a commercially available in vitro packaging kit (e.g. Gigapack Gold available from PromegaBiotech), thereby producing an λ phage particles containing the recombinant λ phage DNAs. The obtained λ phage particles are transfected into host cells, e.g. E. coli, in accordance with a conventional method [Maniatis, T., et al.: Molecular Cloning. A Laboratory Manual, 2nd ed., Cold Spring Harbor Laboratory Press, 1989]. The obtained transfectants are allowed to proliferate, thereby a phage cDNA library can be obtained.

On the other hand, in regard to a group of the recombinant plasmid DNAs, for example, plasmid DNAs are transformed into host cells, e.g. E. coli in accordance with a conventional method. The obtained transformant is allowed to proliferate, thereby a plasmid cDNA library can be obtained.

Next, the above transformants are allowed to proliferate and transferred onto a nylon membrane or a nitrocellulose membrane , e.g. Gene Screening Plus available from DuPont, and proteins are removed under a alkaline condition. The λ phage DNAs or plasmid DNAs on the membrane are hybridized with a radioactive-labeled probe derived from a partial fragment of a DNA encoding Mre11 protein, then positive clones are selected. Herein, the probe to be used can be prepared by PCR using oligonucleotide primers designed based on a nucleotide sequence of a DNA encoding a partial amino acid sequence of Mre11 protein. For example, for PCR-preparation of a probe to be used for screening clones including a DNA encoding yeast Mre11 protein, 5′-gtgccattattatttcagaa-3′ (SEQ ID NO: 9) and 5′-gggatcaagtacaactattttc-3′ (SEQ ID NO: 10) may be used as a primer. Then, the obtained positive clones are cultured, and phage DNAs or plasmid DNAs are prepared from the culture.

In addition, for preparing a DNA encoding Mre11 protein by direct PCR-amplification, initially genome DNA or cDNA are prepared from cells of the organisms described in section (1) above. Then, using the obtained genome DNA or cDNA as templates, a DNA encoding an amino acid sequence at N-terminal side of the Mre11 protein and a DNA encoding an amino acid sequence at C-terminal side thereof are used as primers, PCR can be performed. Next, a PCR-amplified fragment is ligated to a suitable vector. Examples of primers to be used for amplifying DNA encoding yeast Mre11 protein include 5′-atggactatcctgatccaga-3′ (SEQ ID NO: 11) and 5′-gggatcaagtacaactattttc-3′ (SEQ ID NO: 12). In the case of obtaining the DNA encoding the Mre11 protein with direct PCR-amplification, pfu polymerase is preferably used as a DNA sythetase due to its infrequent misreading.

The DNA obtained according to the above method is verified to be a DNA encoding the Mre11 protein through determination of a nucleotide sequence by any of known methods, e.g. the dideoxy method. Determination of a nucleotide sequence can usually be carried out by use of an automatic nucleotide sequencer (e.g. 373A DNA sequencer manufactured by PERKIN-ELMER).

In SEQ ID NOS: 1 and 3, nucleotide sequences of the DNAs encoding the Mre11 proteins derived from Saccharomyces cerevisiae and human, respectively, and in SEQ ID NOS: 2 and 4, the amino acid sequences of the proteins thereof are exemplified. However, as long as the protein has physiological activity of Mre11 protein, there may occur variations such as deletions, substitutions, and additions of one or more amino acids relative to these amino acid sequences.

For example, one or more, preferably about 20 to 30, more preferably 10 to 20 amino acids may be deleted from the amino acid sequence represented by SEQ ID NOS: 2 or 4. Or one or more, preferably about 20 to 30, more preferably 10 to 20 amino acids may be added to the amino acid sequence represented by SEQ ID NOS: 2 or 4. Alternatively, one or more, preferably about 20 to 30, more preferably 10 to 20 amino acids may be substituted by other amino acid(s) in the amino acid sequence represented by SEQ ID NOS: 2 or 4.

Further, a DNA which can hybridize with the above-mentioned gene under a stringent condition, as long as it encodes a protein having physiological activity of Mre11 protein, can be used in the present invention. The term “a stringent condition” means that, for example, the sodium concentration is from 33 to 70 mM, preferably 50 to 66 mM and the temperature is from 40 to 70° C., preferably 55 to 68° C.

The physiological activity of Mre11 protein means, as mentioned above, double-stranded DNA binding activity, double-stranded DNA exonuclease activity and single-stranded DNA endonuclease activity, and each of these activities can be determined in accordance with a method of Furuse et al. [Furuse et al.: EMBO J 17:6412-25 (1998)]

2. Introduction of Variations into a DNA Encoding Foreign Mre11 Protein

When a DNA encoding a variant of foreign Mre11 protein with modification in its nuclease domain, is introduced into a cell in a state such that the DNA can be expressed, the telomeres are shortened more than in the case where a DNA encoding a wild-type Mre11 protein is introduced. However, when a DNA encoding a variant of foreign Mre11 protein with modification in its C-terminal domain, is introduced in a state such that the DNA can be expressed, the telomere length is retained or increased, differing from the case where a DNA encoding a wild-type Mre11 protein is introduced. Here the term “nuclease domain” means, in an amino acid sequence constituting the Mre11 protein, an amino acid sequence responsible for double-stranded DNA exonuclease activity and single-stranded DNA endonuclease activity of the protein. In particular, for example with respect to yeast Mre11 protein, the nuclease domain corresponds to a region from 225th to 450th amino acid numbered from the N-terminal. The term “C-terminal domain” means, in an amino acid sequence constituting the Mre11 protein, an amino acid sequence at carboxyl terminal region responsible for double-stranded DNA binding activity of the protein. In particular, for example with respect to yeast Mre11 protein, the C-terminal domain corresponds to a region from 600th amino acid onwards numbered from the N-terminal.

A DNA encoding a variant of Mre11 protein can be prepared as follows. For example, for preparing a DNA encoding a protein having specific one or more amino acid in the nuclease domain of the Mre11 protein substituted by different amino acid(s), there can be employed for example a site-directed mutagenesis method based on PCR [Saigo Kaoru et al.: “BUNSHISEIBUTSU-GAKU JIKKEN PROTOCOL I (Experiment Protocol I for Molecular Biology), p. 263 -270, Maruzen, 1997], which is known in the technical field of the present invention.

3. Introduction of a DNA Encoding Mre11 Protein or a Variant of Mre11 Protein into a Cell

A DNA encoding Mre11 protein or a variant of Mre11 protein can be introduced into a cell as mentioned below. That is, after preparing a recombinant vector which includes a DNA encoding the Mre11 protein or variant tehreof obtained in section 1 or 2 above, the obtained recombinant vector is introduced into a host cell.

(1) Preparation of a Recombinant Vector

The recombinant vector can be obtained by ligating (inserting) to a suitable vector a DNA encoding the Mre11 protein or the variant of Mre11 protein. Herein, the vector to be used is not particularly limited, as long as it is replicable in the host cell. Examples thereof include a plasmid vector and a virus vector.

A plasmid vector for a yeast host cell may be pMAC561aur, YEp13, YEp24, YCp50 etc., and a virus vector for an animal host cell may be a retrovirus, a vaccinia virus and the like.

For ligating the DNA encoding the Mre11 protein or the variant of Mre11 protein to the vector, there may be employed a method wherein initially a DNA fragment including the above DNA is cleaved by (an) appropriate restriction enzyme(s), and then it is ligated to (a) restriction site(s) or a multi cloning site in the vector DNA.

It is necessary that the DNA encoding the Mre11 protein or variant of Mre11 protein should be incorporated into the vector in a state such they can be expressed in the host cell. Thus, in addition to a promoter and the DNA encoding the Mre11 protein or the variant of Mre11 protein, a cis element such as an enhancer, a splicing signal, a poly A addition signal and a selective marker can be ligated as desired. Here, examples of the selective marker include a dihydrofolate reductase gene, a blasticidin resistant gene, an aureobasidin resistant gene, and a G418 resistant gene.

Incidentally, a recombinant vector pMACMre11D16A including a DNA encoding Mre11 protein having the asparatic acid substituted by alanine at the 16th amino acid position from an N-terminal of the Mre11 protein, and a recombinant vector pMACMre11 ΔC49 including a DNA encoding Mre11 protein having a C-terminal domain deleted each have been introduced into a E coli DH5α strain (ERKNMRE11D 16AYOPAUR and ERKNMRE11DC49YOPAUR) and deposited on Dec. 22, 1999, at the National Institute of Bioscience and Human-Technology, the National Institute of Advance Industrial Science and Technology, under the Ministry of Economy, Trade and Industry, (Higashi 1-1-3, Tsukuba, Ibaraki, Japan) under the accession Nos. FERM BP-7421 and FERM BP-7422.

(2) Preparation of a Transformant Cell

Preparation of a transformant cell can be carried out by introducing the recombinant vector into a host such that a gene of interest can be expressed. Herein, the host may be, without particularly limitation, any eukaryotic cell as long as it has a filamentous chromosome including telomeres. Examples thereof include yeasts such as Saccharomyces cerevisiae and Schizosaccharomyces pombe, animal cells such as COS cells and CHO cells, or an insect cells such as Sf9, Sf21.

When using a yeast as a host, Saccharomyces cerevisiae, Schizosaccharomyces pombe, Pichia pastoris etc. may be used. In this case, any promoter may be used, without particularly limitation, that can be expressed in the yeast. Examples thereof include a gal1 promoter, a gal10 promoter, a heat shock protein promoter, a MFα1 promoter, a PH05 promoter, a PGK promoter, a GAP promoter, an ADH promoter, an AOX1 promoter. The method of introducing the recombinant vector into the yeast may be any of methods by which a DNA can be introduced into the yeast, without particular limitation, for example, electroporation [Becker, D. M. et al.: Methods. Enzymol., 194: 182(1990)], spheroplast method [Hinnen, A. et al.: Proc. Natl. Acad. Sci., USA, 75:1929(1978)], and lithium acetate method [Itoh, H.: J. Bacteriol., 153: 163 (1983)].

When using an animal cell as a host, a monkey COS-7 cell, Vero, a Chinese hamster ovary cell (CHO cell), a mouse L cell, a rat GH3, or a human FL cell can be used. As a promoter, an SRα promoter, an SV40 promoter, an LTR promoter, a CMV promoter or the like may be used, and an human cytomegalovirus early gene promoter etc. may be used as well. Exemplary methods for introducing the recombinant vector into the animal cell include electroporation, the lithium acetate method and the lipofection method.

When using an insect cell as a host, an Sf9 cell, an Sf21 cell or the like may be used. Exemplary methods for introducing the recombinant vector into the insect cell include the calcium phosphate method, the lipofection method, electroporation and the like.

4. Analysis of Telomere Length

Telomere length can be analyzed by Southern hybridization using a probe which specifically hybridizes to a telomere sequence. For example, for analyzing the telomere length in the yeast cells, initially the genome DNAs are extracted from control yeast cells and test yeast cells. Next, the obtained genome DNAs are digested by (an) appropriate restriction enzyme(s) (e.g. XhoI), and then the DNA fragments are isolated by agarose gel electrophoresis. Thereafter, they are transcribed onto a nylon membrane under an alkaline condition, and they are hybridized to a labeled-probe which specifically hybridizes with a telomere sequence under an appropriate condition. Herein, the probe may be an oligonucleotide comprising a sequence of 5′-gtgtgtgtgtgtgtgtgtgt-3′ (SEQ ID NO: 13). After washing the membrane, they are visualized with autoradiography or imaging plate (e.g. BAS2000 manufactured by Fuji Photo Film Co., Ltd.). Comparison between lanes of the control yeast cells and lanes of the test yeast cells showed clearly the difference in their telomere length.

5. Application as an Agent for Controlling Telomere Length and a Gene Therapeutic Agent for Telomere Length-Related Diseases

By modifying physiological activity of the endogenous Mre11 protein in a eukaryotic cell, it is possible to control telomere length in the cell. Therefore, an agent comprising as an active component a substance which modifies physiological activity of endogenous Mre11 protein in a eukaryotic cell, is valuable as a control agent for telomere length and a gene therapeutic agent. Examples of the substance which modifies physiological activity of endogenous Mre11 protein in a eukaryotic cell, include a DNA construct comprising a DNA encoding Mre11 protein or a DNA encoding a protein wherein the nuclease domain or the C-terminal domain of the Mre11 protein is modified in a state such that the DNA can be expressed.

(1) An Agent for Controlling Telomere Length

A DNA construct comprising a DNA encoding the Mre11 protein a protein wherein the nuclease domain or the C-terminal domain of the Mre11 protein is modified in a state such that the DNA can be expressed, is useful as a agent for controlling the telomere length (e.g. a reagent for telomere length control). That is, it is possible to control telomere length in any cell derived from any organism having a chromosome containing telomeres. To facilitate shortening of telomeres, a DNA construct comprising a DNA encoding the Mre11 protein or a protein having modified a nuclease domain of the nuclease domain of the Mre11 protein in a state such that either DNA can be expressed, is introduced into the cell. In contrast, to retain or increase telomere length, a DNA construct comprising a DNA encoding a protein wherein the C-terminal domain of the Mre11 protein is modified in a state such that the DNA can be expressed, is introduced into the cell.

The method of introducing a gene into a yeast cell, may be any method without particular limitation as long as it is a method of introducing a DNA into a yeast, and examples thereof include electroporation [Becker, D. M. et al.: Methods. Enzymol., 194: 182(1990)], spheroplast method [Hinnen, A. et al.: Proc. Natl. Acad. Sci., USA, 75:1929(1978)], and lithium acetate method [Itoh, H.: J. Bacteriol., 153: 163 (1983)]. Exemplary methods of introducing a gene into an animal cell include electroporation, calcium phosphate method, and lipofection method.

(2) Gene Therapeutic Agent for Telomere Length-Associated Diseases

The term “telomere length-associated diseases” means any disease which relates to telomere length for the onset thereof. Examples thereof include malignant tumors (e.g. melanoma, hepatoma, breast cancer, gastric cancer, brain tumor) in which shortening of telomeres in their chromosomes does not occur because of their increased telomerase activity, and senile diseases wherein shortening of telomeres is enhanced compared with a normal cell. When a DNA construct comprising a DNA encoding the Mre11 protein or a DNA encoding a protein wherein the nuclease domain or the C-terminal domain of the Mre11 protein. is modified in a state such that the DNA can be expressed, is used as the gene therapeutic agent for telomere length-associated diseases, the DNA construct may be directly administered to a subject by methods such as injection, gene gun etc. Examples of vector which can be used for the DNA construct, include an adenovirus vector, an adeno-associated virus vector, a herpes virus vector, a vaccinia virus vector, a retrovirus vector etc. These virus vectors enable the DNA to be more efficiently introduced into the cell. Further, a method may be employed which comprises introducing the DNA construct into a phospholipid vesicle such as liposome and administering the liposome to a subject. In this method, since the liposome is a closed vesicle including a bio-degradable material, mixing the liposome with the gene of the present invention allows the gene hold in the lipid bilayer or the internal aqueous layer of the liposome (liposome-gene complex). Next, by culturing a cell in the presence of the complexes the gene in the complex is incorporated into the cell (lipofection method). Then, the obtained cell may be administered to a subject according to administration methods described below.

As an administration form of the gene therapeutic agent of the present invention, there may be employed local administration into an epithelical tissue (epidermis) or any of various organs or tissues etc. in addition to general systemic administration e.g. intravenous or intraarterial administration etc.. Moreover, an administration form in combination with catheter technique, surgical operation etc. can be employed.

The dose of the gene therapeutic agent of the present invention is varied, depending on the age, the sex, the condition of the patient, the route or the frequency of administration, or the formulation type, but usually the range from 0.1 to 100 mg/body/day of the gene of the present invention is appropriate for adults.

### BRIEF DESCRIPTION OF THE DRAWINGS

FIG. 1 is a map of a structure of a recombinant vector.

FIG. 2 is a schematic view of an arm of an yeast chromosome.

FIG. 3 is a photograph of gel electropolation showing telomere length of a yeast cell introduced with a recombinant vector .

### BEST MODE FOR CARRYING OUT THE INVENTION

The present invention will hereinafter be described in detail with reference to Examples, but the scope of the present invention is not limited thereto.

### EXAMPLE 1

Preparation of a DNA Recombinant Vector Encoding a Yeast Mre11 Protein and a Variant Yeast Mre11 Protein

(1) Preparation of a DNA Encoding Mre11 Protein

E. coli cells containing a genomic DNA library of yeast DBY939 strain constructed by using a YEp24 yeast—E. coli shuttle vector (constructed by M. Carlson) were plated onto LB agar medium containing ampicillin, and the obtained colonies were transfer to a nylon membrane, then the DNA derived from the colonies onto the membrane in accordance with a conventional method. The polymerase-chain-reaction (PCR) was performed with oligonucleotides as primers, each of which has a sequence corresponding to the DNA sequence encoding the N-terminal region and the C-terminal region of the MRE11 protein respectively, and the genome DNA of wild-type blast yeast strain ORD149 as a template. The obtained DNA fragment was cloned in a vector for E. coli, and the fragment excised from the vector was labeled using [³²P]-dCTP. This probe was contacted with the nylon membrane prepared previously under stringent conditions for hybridization, thereby identifying a colony containing a fragment of Mre11 gene. This colony was isolated, and the scolony hybridization was repeated similarly to above to obtain a single clone containing the Mre11 gene fragment. From this plasmid DNA, the Mre11 fragment was excised by restriction enzyme tdigestion and cloned into another vector compatible for E. coli, thus obtaining a plasmid pMRE11tr.

(2) Preparation of a DNA Encoding a Variant of Mre11 Protein

Using the DNA encoding the Mre11 protein obtained in (1) above, in accordance with a method of Hashimoto-Gotoh et al. [Hashimoto-Gotoh, T., et al.: Gene, 152:271-275 (1995)], DNAs encoding the following proteins were prepared: a protein (also referred to as D16A type Mre11 protein) wherein the C-terminal domain of the Mre11 protein was deleted, a protein (also referred as to DC49 type Mre11 protein) wherein the asparatic acid at 16th position from the N-terminal of the Mre11 was substituted by alanine, and a protein wherein the C-terminal domain of the Mre11 protein was deleted and the asparatic acid at 16th position from an N-terminal of the Mre11 was substituted by alanine. That is, one nucleotide substitution from gat to gct of the codon at 16th position (47th nucleotide was changed from a to c)was carried out for D16A variant, and further the 643rd and 644th codons were changed from gct agt to gcc tag to introduce a termination codon, for DC49 type variant.

(3) Construction of a Recombinant Vector Containing a DNA Encoding Mre11 Protein or a Variant of Mre11 Protein

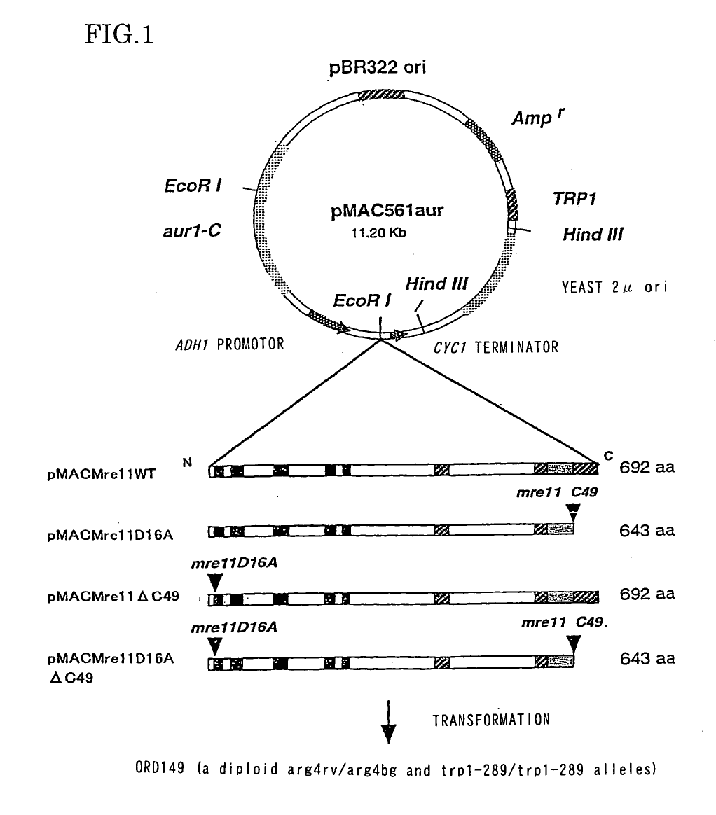

The DNA fragments obtained in (1) and (2) above which encode Mre11 protein and a variant of Mre11 protein, as shown in FIG. 1, were inserted at an EcoRI site present between ADH1 promoter and CYC1 terminator in a yeast-E. coli shuttle vector including an aureobasidin resistant gene, thereby constructing a vector which enables high level expression in yeast. The recombinant vector containing a DNA encoding wild type Mre11 protein was named as pMACMre11WT, the recombinant vector containing a DNA encoding Mre11 protein with the asparatic acid at 16th position from the N-terminal substituted by alanine was as pMACMre11D16A, the recombinant vector containing a DNA encoding Mre11 protein deleted a C-terminal domain was as pMACMre11ΔC49, and the recombinant vector including a DNA encoding Mre11 protein wherein the asparatic acid at the 16th position from N-terminal was substituted by alanine and C-terminal domain was deletedwas as pMACMre11D16AΔC49.

### EXAMPLE 2

Transformation of Yeast with a Recombinant Vector

Transformation of yeast Saccharomyces cerevisiae ORD149 strain (a diploid having arg4rv/arg4bg and trp1-289/trp1-289 alleles), which was given by Dr. Alain Nicolas (Institut Curie, France). with the recombinant vector obtained in Example 1 was conducted, as follows. The yeast strain was washed with 1M sorbitol and mixed with the vector. After the DNA was introduced into the yeast cells by electroporation, the cells (the yeast strain having mutation in trp1 gene and the vector having a selectable marker TRP1 gene) were allowed to grow on a tryptophan-free minimal agar medium containing 1M sorbitol (, and the colonies having the plasmid were selected. These yeast colonies were further grown in a medium containing 0.2 μg/ml of aureobasidin.

### EXAMPLE 3

Analysis of Telomere Length

The telomere length of each transformant obtained in Example 2 was analyzed as follows. That is, a yeast cell including only pMAC561aur vector and transformant yeast cells which were obtained in Example 2, each having one of the recombinant vectors, pMACMre11WT, pMACMre11D16A, pMACMre11ΔC49, and pMACMre11D16AΔC49 were cultured for 12-16 hours in a nutrient medium containing 0.2 μg/ml of aureobasidin, and the cells were harvested. Next, in accordance with a conventional method, genomic DNA was extracted from the cell, and after digestion by a restriction enzyme XhoI, the DNA fragments were separated by agarose gel electrophoresis. The DNA fragments on the agarose gel were transferred onto a nylon membrane with vacuum under an alkaline condition, and the membrane was analyzed by Southern hybridization according to a method of Church et al. [Church, G. M. et. al.: Proc. Natl. Acad. Sci. U.S.A., 81: 1991-1995(1984)]. As a probe, an oligonucleotide labeled with a radioactive isotope p³² at 5′-endwhich comprises 20 nucleotides of 5′-gtgtgtgtgtgtgtgtgtgt-3′ (SEQ ID NO: 13) was used. The hybridization was performed for 20 hours at 50° C. After washing, the radioactive signals on the membrane were visualized by autoradiography or with an imaging plate manufactured by Fuji Photo Film Co., Ltd.

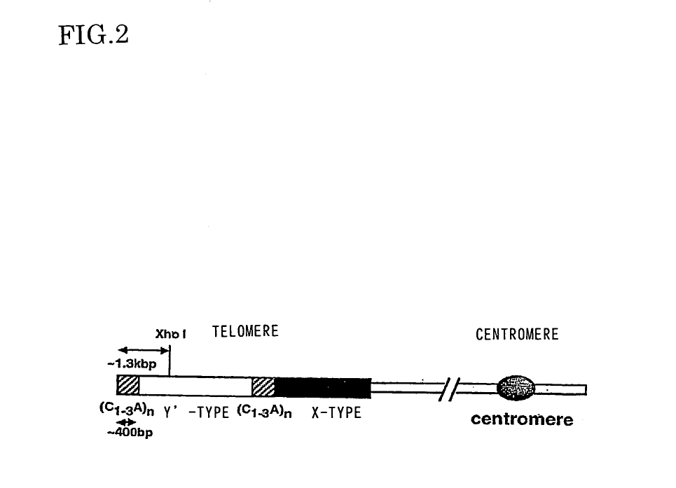

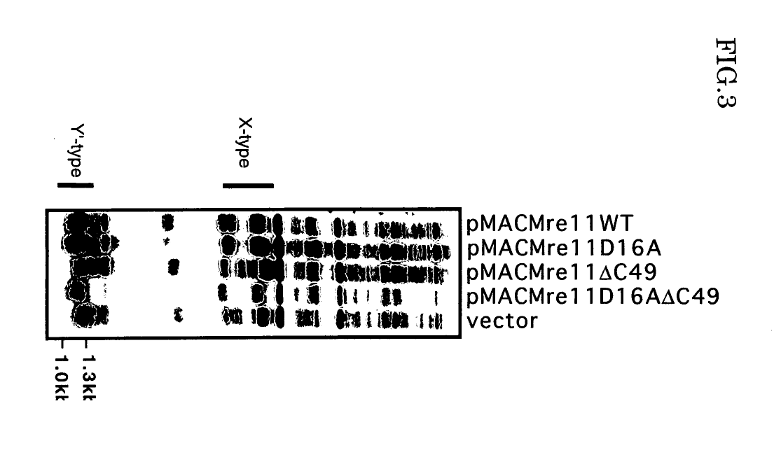

A chromosome of the obtained yeast has a structure as shown in FIG. 2. A region with which the probe can hybridize exists at some midpoints (X type) in addition to telomeres at the chromosome terminals (Y′ type). In comparison with the yeast carrying only a vector not containing the DNA encoding Mre11 protein, when wild type Mre11 protein or the D16A type Mre11 protein with a mutation in the nuclease domain was highly expressed, the shortening of telomeres in the telomere regions of both X and Y types was observed (FIG. 3) In particular, the yeast in which the D16A type Mre11 protein with a mutation in the nuclease domain was highly expressed, the shortening of telomeres was remarkable. In contrast, with regard to a cell line in which the DC49 type Mre11 protein deleted the C-terminal region was highly expressed, the elongation of telomeres was observed in all telomere regions of X and Y types. In view of the forgoing, it is shown that telomeres can be elongated by introducing into a cell a DNA encoding a protein having the deletion of C-terminal domain of Mre11 protein, and the telomeres can be shortened by introducinga DNA encoding Mre11 protein.

### INDUSTRIAL APPLICABILITY

According to the present invention, there are provided a method of controlling telomere length, a control agent for controlling telomere length, and a gene therapeutic agent for telomere length-associated disease.

Sequence List Free Text

SEQ ID NO: 9: synthesis DNA
SEQ ID NO: 10: synthesis DNA
SEQ ID NO: 11: synthesis DNA
SEQ ID NO: 12: synthesis DNA
SEQ ID NO: 13: synthesis DNA

1. A method of controlling telomere length wherein the method comprises modifying physiological activity of endogenous Mre11 protein in a eukaryotic cell.

2. The method of controlling telomere length according to claim 1, wherein the modification of physiological activity of the endogenous Mre11 protein is carried out by introducing into a cell a DNA encoding foreign Mre11 protein or a DNA encoding a protein wherein the nuclease domain or the C-terminal domain of the foreign Mre11 protein is modified, in a state such that the DNA can be expressed.

3. The method of controlling telomere length according to claim 2, wherein the foreign Mre11 protein is the following protein (a) or (b):
(a) a protein comprising an amino acid sequence represented by SEQ ID NO: 2 or 4; or
(b) a protein comprising an amino acid sequence which is the amino acid sequence represented by SEQ ID NO: 2 or 4 from, in, or to which one or more amino acids are deleted, substituted or added, and having physiological activity of Mre11 protein.

4. The method of controlling telomere length according to claim 2, wherein the DNA encoding the foreign Mre11 protein is the following DNA (c) or (d):
(c) a DNA comprising a nucleotide sequence represented by SEQ ID NO: 1 or 3; or
(d) a DNA which can hybridize with the DNA of (c) under a stringent condition and encodes a protein having physiological activity of Mre11 protein.

5. The method of controlling telomere length according to claim 2, wherein the protein having the modified nuclease domain of the foreign Mre11 protein is the following protein (e) or (f):
(e) a protein comprising an amino acid sequence represented by SEQ ID NO: 6; or
f) a protein comprising an amino acid sequence which is the amino acid sequence represented by SEQ ID NO: 6 from, in, or to which one or more amino acids are deleted, substituted or added, and having physiological activity (except nuclease activity) of Mre11 protein.

6. The method of controlling telomere length according to claim 2, wherein the DNA encoding the protein having the modified nuclease domain of the foreign Mre11 protein is the following DNA (g) or (h):
(g) a DNA comprising a nucleotide sequence represented SEQ ID NO: 5; or
(h) a DNA which can hybridize with the DNA of (g) under a stringent condition and encodes a protein having physiological activity (except nuclease activity) of Mre11 protein.

7. The method of controlling telomere length according to claim 2, wherein the protein having the modified C-terminal domain of the foreign Mre11 protein is the following protein (i) or (j):
(i) a protein comprising an amino acid sequence represented by SEQ ID NO: 8; or
(j) a protein comprising an amino acid sequence which is the amino acid sequence represented by SEQ ID NO: 8 from, in or to which one or more amino acids are deleted, substituted or added, and having physiological activity (except double-stranded DNA binding activity) of Mre11 protein.

8. The method of controlling telomere length according to claim 2, wherein the DNA encoding the protein having the modified C-terminal domain of the foreign Mre11 protein is the following DNA (k) or (1):
(k) a DNA comprising a nucleotide sequence represented by SEQ ID NO: 7; or
(l) a DNA which can hybridize with the DNA of (k) and encodes a protein having physiological activity (except double-stranded DNA binding activity) of Mre11 protein.

9. An agent for controlling telomere length comprising as an active component a substance which modifies physiological activity of endogenous Mre11 protein in a eukaryotic cell.

10. The agent for controlling telomere length according to claim 9, wherein the substance is a DNA construct which comprises a DNA encoding a foreign Mre11 protein or a DNA encoding a protein wherein the nuclease domain or the C-terminal domain of the Mre11 protein is modified in a state such that the DNA can be expressed.

11. The agent for controlling telomere length according to claim 10, wherein the foreign Mre11 protein is the following protein (a) or (b):
(a) a protein comprising an amino acid sequence represented by SEQ ID NO: 2 or 4; or
(b) a protein comprising an amino acid sequence which is the amino acid sequence represented by SEQ ID NO: 2 or 4 from, in, or to which one or more amino acids are deleted, substituted or added, and having physiological activity of Mre11 protein.

12. The agent for controlling telomere length according to claim 10, wherein the DNA encoding the foreign Mre11 protein is the following DNA (c) or (d):
(c) a DNA comprising a nucleotide sequence represented by SEQ ID NO: 1 or 3; or
(d) a DNA which can hybridize with the DNA of (c) under a stringent condition and encodes a protein having physiological activity of Mre11 protein.

13. The agent for controlling telomere length according to claim 10, wherein the protein having the modified nuclease domain of the foreign Mre11 protein is the following protein (e) or (f):
(e) a protein comprising an amino acid sequence represented by SEQ ID NO: 6; or
(f) a protein comprising an amino acid sequence which is the amino acid sequence represented by SEQ ID NO: 6 from, in, or to. which one or more amino acids are deleted, substituted or added, and having physiological activity (except nuclease activity) of Mre11 protein.

14. The agent for controlling telomere length according to claim 10, wherein the DNA encoding the protein having the modified nuclease domain of the foreign Mre11 protein is the following DNA (g) or (h):
(g) a DNA comprising a nucleotide sequence represented SEQ ID NO: 5; or
(h) a DNA which can hybridize with the DNA of (g) under a stringent condition and encodes a protein having physiological activity (except nuclease activity) of Mre11 protein.

15. The agent for controlling telomere length according to claim 10, wherein the protein having the modified C-terminal domain of the foreign Mre11 protein is the following protein (i) or ():
(i) a protein comprising an amino acid sequence represented by SEQ ID NO: 8; or
(j) a protein comprising an amino acid sequence which is the amino acid sequence represented by SEQ ID NO: 8 from, in or to which one or more amino acids are deleted, substituted or added, and having physiological activity (except double-stranded DNA binding activity) of Mre11 protein.

16. The agent for controlling telomere length according to claim 10, wherein the DNA encoding the protein having the modified C-terminal domain of the foreign Mre11 protein is the following DNA (k) or (1):
(k) a DNA comprising a nucleotide sequence represented by SEQ ID NO: 7; or
(l) a DNA which can hybridize with the DNA of (g) and encodes a protein having physiological activity (except double-stranded DNA binding activity) of Mre11 protein.

17. A gene therapeutic agent for a telomere length-associated disease comprising as an active agent a substance which modifies physiological activity of endogenous Mre11 protein in a eukaryotic cell.

18. The gene therapeutic agent according to claim 17, wherein the substance is a DNA construct which comprises a DNA encoding foreign Mre11 protein or a DNA encoding a protein wherein the nuclease domain or the C-terminal domain of the Mre11 protein is modified, in a state such that the DNA can be expressed.

19. The gene therapeutic agent according to claim 17, wherein the telomere length-associated disease is a malignant tumor or a cell senescent disease.

20. The gene therapeutic agent according to claim 19, wherein the malignant tumor is at least one selected from the group consisting of melanoma, hepatoma breast cancer, gastric cancer, and brain tumor.

21. The gene therapeutic agent according to claim 18, wherein the foreign Mre11 protein is the following protein (a) or (b):
(a) a protein comprising an amino acid sequence represented by SEQ ID NO: 2 or 4; or
(b) a protein comprising an amino acid sequence which is the amino acid sequence represented by SEQ ID NO: 2 or 4 from, in, or to which one or more amino acids are deleted, substituted or added, and having physiological activity of Mre11 protein.

22. The gene therapeutic agent according to claim 18, wherein the DNA encoding the foreign Mre11 protein is the following DNA (c) or (d):
(c) a DNA comprising a nucleotide sequence represented by SEQ ID NO: 1 or 3; or
(d) a DNA which can hybridize with the DNA of (c) under a stringent condition and encodes a protein having physiological activity of Mre11 protein.

23. The gene therapeutic agent according to claim 18, wherein the protein having the modified nuclease domain of the foreign Mre11 protein is the following protein (e) or (f):
(e) a protein comprising an amino acid sequence represented by SEQ ID NO: 6; or
(f) a protein comprising an amino acid sequence which is the amino acid sequence represented by SEQ ID NO: 6 from, in, or to which one or more amino acids are deleted, substituted or added, and having physiological activity (except nuclease activity) of Mre11 protein.

24. The gene therapeutic agent according to claim 18, wherein the DNA encoding the protein having the modified nuclease domain of the foreign Mre11 protein is the following DNA (g) or (h):
(g) a DNA comprising a nucleotide sequence represented SEQ ID NO: 5; or
(h) a DNA which can hybridize with the DNA of (g) under a stringent condition and encodes a protein having physiological activity (except nuclease activity) of Mre11 protein.

25. The gene therapeutic agent according to claim 18, wherein the protein having the modified C-terminal domain of the foreign Mre11 protein is the following protein (i) or ():
(i) a protein comprising an amino acid sequence represented by SEQ ID NO: 8; or
(j) a protein comprising an amino acid sequence which is the amino acid sequence represented by SEQ ID NO: 8 from, in or to which one or more amino acids are deleted, substituted or added, and having physiological activity (except double-stranded DNA binding activity) of Mre11 protein.

26. The gene therapeutic agent according to claim 18, wherein the DNA encoding the protein having the modified C-terminal domain of the foreign Mre11 protein is the following DNA (k) or (1):
(k) a DNA comprising a nucleotide sequence represented by SEQ ID NO: 7; or
(l) a DNA which can hybridize with the DNA of (g) and encodes a protein having physiological activity (except double-stranded DNA binding activity) of Mre11 protein.


SEQUENCE LISTING (13 sequences)

SEQ ID NO: 1 - DNA, 2079 bp, Saccharomyces cerevisiae
  CDS (1)..(2076)
atggactatcctgatccagacacaataaggattttaattactacagataatcatgtgggttacaacgaaaatgatcccattactggcgatgattcttggaaaactttccatgaagtcatgatgctggccaaaaataacaacgtagacatggttgtacagtccggtgatctttttcacgtgaataagccttccaagaagtcactctaccaagtactgaaaactttgagattatgttgcatgggtgacaagccttgcgagttagaattattgagcgatccctcacaagtttttcactacgatgaatttaccaacgttaactatgaggaccccaactttaatatttctattcccgtattcggcatatcaggtaatcatgatgatgcgtcgggggactcactgttgtgtcctatggatatacttcatgcgactggtctaataaatcatttcgggaaagtcatcgaatctgataaaataaaagtcgtgccattattatttcagaaagggtccactaagttagcattgtacggattagccgctgttcgtgatgaaaggttatttagaacttttaaggatggtggtgtcacttttgaagtaccgactatgcgagaaggtgaatggtttaatttaatgtgcgtccatcaaaatcatacaggtcacacgaatactgcatttttacctgaacagttcttgccagatttcctggatatggtgatatggggtcatgaacatgagtgtattccgaatctcgtacacaatccaattaaaaattttgatgtattacaacctggttcatctgtagctacttcactttgtgaggctgaggcacaacccaagtatgtcttcatccttgacataaagtatggagaagcaccaaaaatgacacctattcctcttgagactatacggacattcaaaatgaaatccatttcgttacaagatgttccccatttgaggcctcacgataaagatgctacgtctaagtatcttattgaacaagttgaagaaatgatccgcgacgctaatgaggaaactaaacaaaaattagcggacgatggtgaaggtgacatggttgcggaattaccgaaaccattgatcagattacgtgttgattatagtgcaccctccaatacacaatcctcaatagattaccaagtagaaaacccgcgtagatttagcaatcgatttgtgggacgtgttgctaacggtaataacgttgtgcagttttataaaaaaaggtcacctgtaactagatcaaaaaaatccggtataaatggaacaagcatcagtgatagagatgttgagaaacttttcagcgaaagtggcggtgaactagaagttcaaactttggttaatgatctcttgaacaaaatgcaactatctttattaccagaagttggtttgaatgaagcagtaaagaagtttgtagataaagatgagaaaacagctcttaaagaatttattagccatgaaatatcgaacgaagttggaatattatctacgaatgaagaatttctgagaacagatgatgcagaggaaatgaaagcgcttataaaacaggttaagcgtgctaacagtgttaggccgactccccctaaagaaaatgatgagacaaatttcgcattcaatggtaatgggctagattccttccggtctagtaatagagaagtaagaactggatctccagacattacccaatcacatgttgataatgaatcaagaataacccatattagtcaagcggaaagcagtaagccaacgagcaaacccaaacgagtgcgaactgcaacgaaaaagaaaattcctgctttttcagactcaactgtcatatccgatgcagaaaatgaactcggtgataataacgatgctcaagatgatgttgatattgatgagaatgacataattatggtcagtactgacgaagaggacgctagttatggtttacttaatggtcgaaaaacaaaaacaaagactcgttctgctgcgagcaccaaaaccgcttccagaaggggaaaaggaagagcatcaaggacgccaaagacggatattcttggaagtctccttgctaagaaaagaaaatag

SEQ ID NO: 2 - protein, 692 aa, Saccharomyces cerevisiae
MDYPDPDTIRILITTDNHVGYNENDPITGDDSWKTFHEVMMLAKNNNVDMVVQSGDLFHVNKPSKKSLYQVLKTLRLCCMGDKPCELELLSDPSQVFHYDEFTNVNYEDPNFNISIPVFGISGNHDDASGDSLLCPMDILHATGLINHFGKVIESDKIKVVPLLFQKGSTKLALYGLAAVRDERLFRTFKDGGVTFEVPTMREGEWFNLMCVHQNHTGHTNTAFLPEQFLPDFLDMVIWGHEHECIPNLVHNPIKNFDVLQPGSSVATSLCEAEAQPKYVFILDIKYGEAPKMTPIPLETIRTFKMKSISLQDVPHLRPHDKDATSKYLIEQVEEMIRDANEETKQKLADDGEGDMVAELPKPLIRLRVDYSAPSNTQSSIDYQVENPRRFSNRFVGRVANGNNVVQFYKKRSPVTRSKKSGINGTSISDRDVEKLFSESGGELEVQTLVNDLLNKMQLSLLPEVGLNEAVKKFVDKDEKTALKEFISHEISNEVGILSTNEEFLRTDDAEEMKALIKQVKRANSVRPTPPKENDETNFAFNGNGLDSFRSSNREVRTGSPDITQSHVDNESRITHISQAESSKPTSKPKRVRTATKKKIPAFSDSTVISDAENELGDNNDAQDDVDIDENDIIMVSTDEEDASYGLLNGRKTKTKTRSAASTKTASRRGKGRASRTPKTDILGSLLAKKRK

SEQ ID NO: 3 - DNA, 2319 bp, Homo sapiens
  CDS (1)..(2316)
atgtcggccacgggaaaggtttctaggccaattcaagttttcacgcgcgcaaactattttttcaaactcatttttcagtttaaagtccgaaaatataactctctcactgtagaacgtattttcataagagccatgtgtggcagtgatgacagttttgacgactttgtaccagatagtcaagagccagcatcttcgcgtacccgaaatcaggatcatctcgacgatgacgaggttccctgctcacaaaggccagatgctgccaacgacactatggttgaagatctcgacgagggagacgagccagctcatgacgaatctgaggacatcatcaaaattctcgttgccactgatattcattgcggttatggagaaaataaagccaacattcatatggatgccgtcaacacttttgaagaagtgcttcaaattgccactgaacaaaaagtcgatatgattttacttggaggagatctttttcatgaaaataatccatctagagaagttcaacatcgtgttacacaattgctccgtcaatattgcttgaatgggaatccaatcgcgttggagtttctctcagatgcttctgtcaattttaatcaaagtgtgtttggtcacgtgaactattacgatcagaatttgaatgtcggtcttccaattttcacaattcatggaaatcatgatgatctatcaggaaaaggacttactgctcttgatcttcttcacgaatctggacttgtaaatctgtttggaaagcacagcaacattcaggagtttattgtttcaccaattttgcttagaaaaggagaaactcgtttggcgttatacggaattggaagtcaaagagatgatcgtcttgttcgagcattcaaaaataatagtatttcatttttgcggccaaacgctggcgccgaggattggtttaatttgtttgtcctccatcaaaatcgtccacggagagccatgcaccgatcgacaggaaactttcttcccgaatctctgattcctcaattctttgaccttcttatttggggacacgaacacgaatgtaaacctgatcctcaatatgttgcttcatcggaagctgttggtgatggattttacattcttcaaccaggatcgactgttgctacttcgctcactccggaagaagccctgcagaaaaatgcattcctaataaagattaaaggaagaaagtttgcttcaaagcctattcctcttcaaacagttcgtccaatggtttgcgatgagcttcttcttgataaaatacctccaggaagtagaccgattttgaaaactgatcgtccaaaacacacggatggccgatacatagatgaaattgccattgaagcgaaaataaatgaaatgattacaacagcgaaagccaagagacgaccacggcaacctgaattacctttgattcgactcaaagtgatctatgatggtgattggctcaatattactccagcaaatgctaaaagaattggtcttcgttatgaaaatgtagtggcaaacgcagtggatatggtttttatcaagaaaaataataagccaaaggagggaaaattacaaaccgaaaacgagaaaaatataactgaaatggccgacgagatgggacaagtttctgcaaccaatttgcaaactatcattaacgactatttcataaaccagcctctagtggatcaaatgacagtgctcaaaccgataggaattggaagagctttagagcaatattcagcaatagaagaaggtggtcttgctacatctgcgaatcgtaactttgatagttgtttgatgcatcaaattgacgttgttcgtaaaacattgaaaaagatgcctctgcctccgattaacgatccatcggatttagacaaattccgtgatctgatcaccaaggatttatacgatatgaaaaaagccgatagcgaaagattcaagaatcctgttgccgatgttgaaatggaagaagatgaagacgatcctcaatactatatgcctccacagtctacttcgagaacaaattatgctagcggcagtgaagatgaagtggcaaattctgatgaagaaatgggaagcagtatctcgagacattcgaagcaacctaccacacgtggtagaggtcgtggtgccagaggagcaggtgcttcacgagaaactactcgtggaagatccaataaagttgtttcaacgcgacaaatcgattcagatggattcgaaattatcaatgattcgccgtctccgcctccagcttcacgatcaaccagaggaaaagcaagaggaaaatcagctccatccaagaaaagggatctaagtttcttctaa

SEQ ID NO: 4 - protein, 772 aa, Homo sapiens
MSATGKVSRPIQVFTRANYFFKLIFQFKVRKYNSLTVERIFIRAMCGSDDSFDDFVPDSQEPASSRTRNQDHLDDDEVPCSQRPDAANDTMVEDLDEGDEPAHDESEDIIKILVATDIHCGYGENKANIHMDAVNTFEEVLQIATEQKVDMILLGGDLFHENNPSREVQHRVTQLLRQYCLNGNPIALEFLSDASVNFNQSVFGHVNYYDQNLNVGLPIFTIHGNHDDLSGKGLTALDLLHESGLVNLFGKHSNIQEFIVSPILLRKGETRLALYGIGSQRDDRLVRAFKNNSISFLRPNAGAEDWFNLFVLHQNRPRRAMHRSTGNFLPESLIPQFFDLLIWGHEHECKPDPQYVASSEAVGDGFYILQPGSTVATSLTPEEALQKNAFLIKIKGRKFASKPIPLQTVRPMVCDELLLDKIPPGSRPILKTDRPKHTDGRYIDEIAIEAKINEMITTAKAKRRPRQPELPLIRLKVIYDGDWLNITPANAKRIGLRYENVVANAVDMVFIKKNNKPKEGKLQTENEKNITEMADEMGQVSATNLQTIINDYFINQPLVDQMTVLKPIGIGRALEQYSAIEEGGLATSANRNFDSCLMHQIDVVRKTLKKMPLPPINDPSDLDKFRDLITKDLYDMKKADSERFKNPVADVEMEEDEDDPQYYMPPQSTSRTNYASGSEDEVANSDEEMGSSISRHSKQPTTRGRGRGARGAGASRETTRGRSNKVVSTRQIDSDGFEIINDSPSPPPASRSTRGKARGKSAPSKKRDLSFF

SEQ ID NO: 5 - DNA, 2079 bp, Saccharomyces cerevisiae
  CDS (1)..(2076)
atggactatcctgatccagacacaataaggattttaattactacagctaatcatgtgggttacaacgaaaatgatcccattactggcgatgattcttggaaaactttccatgaagtcatgatgctggccaaaaataacaacgtagacatggttgtacagtccggtgatctttttcacgtgaataagccttccaagaagtcactctaccaagtactgaaaactttgagattatgttgcatgggtgacaagccttgcgagttagaattattgagcgatccctcacaagtttttcactacgatgaatttaccaacgttaactatgaggaccccaactttaatatttctattcccgtattcggcatatcaggtaatcatgatgatgcgtcgggggactcactgttgtgtcctatggatatacttcatgcgactggtctaataaatcatttcgggaaagtcatcgaatctgataaaataaaagtcgtgccattattatttcagaaagggtccactaagttagcattgtacggattagccgctgttcgtgatgaaaggttatttagaacttttaaggatggtggtgtcacttttgaagtaccgactatgcgagaaggtgaatggtttaatttaatgtgcgtccatcaaaatcatacaggtcacacgaatactgcatttttacctgaacagttcttgccagatttcctggatatggtgatatggggtcatgaacatgagtgtattccgaatctcgtacacaatccaattaaaaattttgatgtattacaacctggttcatctgtagctacttcactttgtgaggctgaggcacaacccaagtatgtcttcatccttgacataaagtatggagaagcaccaaaaatgacacctattcctcttgagactatacggacattcaaaatgaaatccatttcgttacaagatgttccccatttgaggcctcacgataaagatgctacgtctaagtatcttattgaacaagttgaagaaatgatccgcgacgctaatgaggaaactaaacaaaaattagcggacgatggtgaaggtgacatggttgcggaattaccgaaaccattgatcagattacgtgttgattatagtgcaccctccaatacacaatcctcaatagattaccaagtagaaaacccgcgtagatttagcaatcgatttgtgggacgtgttgctaacggtaataacgttgtgcagttttataaaaaaaggtcacctgtaactagatcaaaaaaatccggtataaatggaacaagcatcagtgatagagatgttgagaaacttttcagcgaaagtggcggtgaactagaagttcaaactttggttaatgatctcttgaacaaaatgcaactatctttattaccagaagttggtttgaatgaagcagtaaagaagtttgtagataaagatgagaaaacagctcttaaagaatttattagccatgaaatatcgaacgaagttggaatattatctacgaatgaagaatttctgagaacagatgatgcagaggaaatgaaagcgcttataaaacaggttaagcgtgctaacagtgttaggccgactccccctaaagaaaatgatgagacaaatttcgcattcaatggtaatgggctagattccttccggtctagtaatagagaagtaagaactggatctccagacattacccaatcacatgttgataatgaatcaagaataacccatattagtcaagcggaaagcagtaagccaacgagcaaacccaaacgagtgcgaactgcaacgaaaaagaaaattcctgctttttcagactcaactgtcatatccgatgcagaaaatgaactcggtgataataacgatgctcaagatgatgttgatattgatgagaatgacataattatggtcagtactgacgaagaggacgctagttatggtttacttaatggtcgaaaaacaaaaacaaagactcgttctgctgcgagcaccaaaaccgcttccagaaggggaaaaggaagagcatcaaggacgccaaagacggatattcttggaagtctccttgctaagaaaagaaaatag

SEQ ID NO: 6 - protein, 692 aa, Saccharomyces cerevisiae
MDYPDPDTIRILITTANHVGYNENDPITGDDSWKTFHEVMMLAKNNNVDMVVQSGDLFHVNKPSKKSLYQVLKTLRLCCMGDKPCELELLSDPSQVFHYDEFTNVNYEDPNFNISIPVFGISGNHDDASGDSLLCPMDILHATGLINHFGKVIESDKIKVVPLLFQKGSTKLALYGLAAVRDERLFRTFKDGGVTFEVPTMREGEWFNLMCVHQNHTGHTNTAFLPEQFLPDFLDMVIWGHEHECIPNLVHNPIKNFDVLQPGSSVATSLCEAEAQPKYVFILDIKYGEAPKMTPIPLETIRTFKMKSISLQDVPHLRPHDKDATSKYLIEQVEEMIRDANEETKQKLADDGEGDMVAELPKPLIRLRVDYSAPSNTQSSIDYQVENPRRFSNRFVGRVANGNNVVQFYKKRSPVTRSKKSGINGTSISDRDVEKLFSESGGELEVQTLVNDLLNKMQLSLLPEVGLNEAVKKFVDKDEKTALKEFISHEISNEVGILSTNEEFLRTDDAEEMKALIKQVKRANSVRPTPPKENDETNFAFNGNGLDSFRSSNREVRTGSPDITQSHVDNESRITHISQAESSKPTSKPKRVRTATKKKIPAFSDSTVISDAENELGDNNDAQDDVDIDENDIIMVSTDEEDASYGLLNGRKTKTKTRSAASTKTASRRGKGRASRTPKTDILGSLLAKKRK

SEQ ID NO: 7 - DNA, 1932 bp, Saccharomyces cerevisiae
  CDS (1)..(1929)
atggactatcctgatccagacacaataaggattttaattactacagataatcatgtgggttacaacgaaaatgatcccattactggcgatgattcttggaaaactttccatgaagtcatgatgctggccaaaaataacaacgtagacatggttgtacagtccggtgatctttttcacgtgaataagccttccaagaagtcactctaccaagtactgaaaactttgagattatgttgcatgggtgacaagccttgcgagttagaattattgagcgatccctcacaagtttttcactacgatgaatttaccaacgttaactatgaggaccccaactttaatatttctattcccgtattcggcatatcaggtaatcatgatgatgcgtcgggggactcactgttgtgtcctatggatatacttcatgcgactggtctaataaatcatttcgggaaagtcatcgaatctgataaaataaaagtcgtgccattattatttcagaaagggtccactaagttagcattgtacggattagccgctgttcgtgatgaaaggttatttagaacttttaaggatggtggtgtcacttttgaagtaccgactatgcgagaaggtgaatggtttaatttaatgtgcgtccatcaaaatcatacaggtcacacgaatactgcatttttacctgaacagttcttgccagatttcctggatatggtgatatggggtcatgaacatgagtgtattccgaatctcgtacacaatccaattaaaaattttgatgtattacaacctggttcatctgtagctacttcactttgtgaggctgaggcacaacccaagtatgtcttcatccttgacataaagtatggagaagcaccaaaaatgacacctattcctcttgagactatacggacattcaaaatgaaatccatttcgttacaagatgttccccatttgaggcctcacgataaagatgctacgtctaagtatcttattgaacaagttgaagaaatgatccgcgacgctaatgaggaaactaaacaaaaattagcggacgatggtgaaggtgacatggttgcggaattaccgaaaccattgatcagattacgtgttgattatagtgcaccctccaatacacaatcctcaatagattaccaagtagaaaacccgcgtagatttagcaatcgatttgtgggacgtgttgctaacggtaataacgttgtgcagttttataaaaaaaggtcacctgtaactagatcaaaaaaatccggtataaatggaacaagcatcagtgatagagatgttgagaaacttttcagcgaaagtggcggtgaactagaagttcaaactttggttaatgatctcttgaacaaaatgcaactatctttattaccagaagttggtttgaatgaagcagtaaagaagtttgtagataaagatgagaaaacagctcttaaagaatttattagccatgaaatatcgaacgaagttggaatattatctacgaatgaagaatttctgagaacagatgatgcagaggaaatgaaagcgcttataaaacaggttaagcgtgctaacagtgttaggccgactccccctaaagaaaatgatgagacaaatttcgcattcaatggtaatgggctagattccttccggtctagtaatagagaagtaagaactggatctccagacattacccaatcacatgttgataatgaatcaagaataacccatattagtcaagcggaaagcagtaagccaacgagcaaacccaaacgagtgcgaactgcaacgaaaaagaaaattcctgctttttcagactcaactgtcatatccgatgcagaaaatgaactcggtgataataacgatgctcaagatgatgttgatattgatgagaatgacataattatggtcagtactgacgaagaggacgcctag

SEQ ID NO: 8 - protein, 643 aa, Saccharomyces cerevisiae
MDYPDPDTIRILITTDNHVGYNENDPITGDDSWKTFHEVMMLAKNNNVDMVVQSGDLFHVNKPSKKSLYQVLKTLRLCCMGDKPCELELLSDPSQVFHYDEFTNVNYEDPNFNISIPVFGISGNHDDASGDSLLCPMDILHATGLINHFGKVIESDKIKVVPLLFQKGSTKLALYGLAAVRDERLFRTFKDGGVTFEVPTMREGEWFNLMCVHQNHTGHTNTAFLPEQFLPDFLDMVIWGHEHECIPNLVHNPIKNFDVLQPGSSVATSLCEAEAQPKYVFILDIKYGEAPKMTPIPLETIRTFKMKSISLQDVPHLRPHDKDATSKYLIEQVEEMIRDANEETKQKLADDGEGDMVAELPKPLIRLRVDYSAPSNTQSSIDYQVENPRRFSNRFVGRVANGNNVVQFYKKRSPVTRSKKSGINGTSISDRDVEKLFSESGGELEVQTLVNDLLNKMQLSLLPEVGLNEAVKKFVDKDEKTALKEFISHEISNEVGILSTNEEFLRTDDAEEMKALIKQVKRANSVRPTPPKENDETNFAFNGNGLDSFRSSNREVRTGSPDITQSHVDNESRITHISQAESSKPTSKPKRVRTATKKKIPAFSDSTVISDAENELGDNNDAQDDVDIDENDIIMVSTDEEDA

SEQ ID NO: 9 - DNA, 20 bp, Artificial Sequence
  Description of Artificial Sequencesynthetic DNA
gtgccattattatttcagaa

SEQ ID NO: 10 - DNA, 20 bp, Artificial Sequence
  Description of Artificial Sequencesynthetic DNA
ctggtaataaagatagttgc

SEQ ID NO: 11 - DNA, 20 bp, Artificial Sequence
  Description of Artificial Sequencesynthetic DNA
atggactatcctgatccaga

SEQ ID NO: 12 - DNA, 22 bp, Artificial Sequence
  Description of Artificial Sequencesynthetic DNA
gggatcaagtacaactattttc

SEQ ID NO: 13 - DNA, 20 bp, Artificial Sequence
  Description of Artificial Sequencesynthetic DNA
gtgtgtgtgtgtgtgtgtgt